(19) United States
(12) Patent Application Publication
(10) Pub. No.: US 2013/0344177 A1
(43) Pub. Date: Dec. 26, 2013

(54) WEIGHT LOSS BEVERAGE

(71) Applicants: D. Gary Young, Alpine, UT (US); Marc Schreuder, Provo, UT (US)

(72) Inventors: D. Gary Young, Alpine, UT (US); Marc Schreuder, Provo, UT (US)

(73) Assignee: YL HOLDINGS, INC., Lehi, UT (US)

(21) Appl. No.: 13/624,059

(22) Filed: Sep. 21, 2012

Related U.S. Application Data
(63) Continuation of application No. 13/533,829, filed on Jun. 26, 2012.

(51) Int. Cl. A61K 36/82; A61K 36/898; A61K 36/54; A61P 3/00; A61P 3/04; A61P 3/10; A61P 7/10; A61K 36/324; A61P 29/00

(52) U.S. Cl. 424/729; 424/725; 424/774

(57) ABSTRACT

A beverage composition for promoting weight loss, the beverage including a stimulant agent, a fat burning agent, a carbohydrate blocking agent, an anti-cholesterol agent, an insulin sensitizing agent, a sweetening agent, an aromatic agent, an antioxidant agent, an anti-inflammatory agent, a dietary agent, and an diuretic agent. The present invention further includes methods for administering the beverage composition to promote weight loss and provide energy to the user.

### RELATED APPLICATIONS

The present application claims priority to U.S. patent application Ser. No. 13/533,829, filed Jun. 26, 2012 and titled WEIGHT LOSS BEVERAGE, which is incorporated herein by reference, in its entirety.

### BACKGROUND

The present disclosure relates to a weight loss beverage. Specifically, this disclosure relates to a beverage comprising a premium blend of wholesome and rare ingredients, and which offers a natural alternative to sugar-laden fruit drinks from concentrates, sodas and coffees. As such, the beverage of the present invention may be used as part of a healthy weight loss program.

Weight loss, in the context of medicine, health or physical fitness, can be defined as a reduction of the total body mass, due to a mean loss of fluid, body fat or adipose tissue. Weight loss typically occurs from a conscious effort to improve an actual or perceived overweight or obese state. Therapeutic weight loss, in individuals who are overweight or obese, can also decrease the likelihood of developing diseases such as diabetes, heart disease, high blood pressure, osteoarthritis, and certain types of cancer.

Weight loss occurs when an individual is in a state of negative thermodynamic flux, meaning the body is exerting more energy (i.e. in work and metabolism) than it is consuming (i.e. from food or other nutritional supplements). When this occurs, the body uses stored reserves from fat or muscle, thereby resulting in weight loss.

While brain chemistry and mental conditioning are known to play a role in weight loss, there are many other physical, chemical and emotional factors that contribute to weight loss. For example, a large body of literature shows that many phytonutrients and other natural products are used to treat obesity as metabolic stimulants, appetite suppressants, and starch blockers and by regulation of glucose and lipid metabolism, as well as targeting adipocyte differentiation. Currently available weight loss compositions address some of these factors, but fail to address others. Thus, while compositions currently exist which are designed to produce weight loss, challenges still exist. Accordingly, it would be an improvement in the art to augment or even replace current techniques with other techniques and/or compositions. The present invention addresses and overcomes these challenges.

### BRIEF SUMMARY

The present disclosure relates to a weight loss beverage. Specifically, this disclosure relates to a beverage comprising a premium blend of wholesome and rare ingredients, and which offers a natural alternative to sugar-laden fruit drinks from concentrates, sodas and coffees. As such, the beverage of the present invention may be used as part of a healthy weight loss program.

Some implementations of weight loss beverages may include a stimulant agent, a fat burning and/or blocking agent, a carbohydrate blocking agent, an anti-cholesterol agent, an insulin sensitizing agent, a sweetening agent, an aromatic agent, an antioxidant agent, an anti-inflammatory agent, a dietary fiber, an appetite control agent, and a diuretic agent. Embodiments of weight loss beverage may further include other natural active and inactive ingredients to provide desired aromas, flavors, textures, and colors.

Some implementations may provide a method for administering the weight loss beverage to a user to assist in weight loss. The weight loss beverage may be used as a weight loss supplement or as a main component of a weight loss program. The weight loss beverage may be taken in combination with other weight loss supplements, or may be used alone. The weight loss beverage may further be taken in combination with physical exercise to increase the weight loss benefits.

### DETAILED DESCRIPTION

The present disclosure relates to a weight loss beverage. Specifically, this disclosure relates to a beverage which may include a premium blend of wholesome and rare ingredients, and which offers a natural alternative to sugar-laden fruit drinks from concentrates, sodas and coffees. As such, disclosed beverages may be used as part of a healthy weight loss program.

Throughout this specification, there are ranges defined by upper and lower boundaries. Each lower boundary can be combined with each upper boundary to define a range. The lower and upper boundaries should each be taken as a separate element.

As used herein, the terms “phytonutrient” and “phytochemical” may denote any of various natural, biologically-active compounds found in plants. Thus, these terms may be used interchangeably with the terms “natural,” “naturally occurring,” and “naturally derived” when describing the ingredients and various components of the present invention.

In general, the disclosed embodiments may provide certain formulae and combinations of formulae which promote weight loss. Some aspects of the disclosed embodiments may provide various nutraceutical compositions which may include various essential oils, plants, herbs, extracts, spices, phytochemicals and pigments which have been found to be effective in promoting weight loss. Thus, while the disclosed embodiments may disclose specific examples of known nutraceutical compositions and combined nutraceutical therapies, one having skill in the art will appreciate that the specific components of the disclosed embodiments may be substituted with natural or synthetic equivalents to achieve a desired results within the spirit and teaching of the disclosed embodiments. Accordingly, the specific embodiments provided are not intended to be limiting, but rather are provided examples disclosing and teaching the underlying principles of weight loss beverages and methods of making and using such beverages.

Some embodiments a weight loss beverage may comprising 1) a stimulant agent, 2) a fat blocking agent, 3) a carbohydrate blocking agent, 4) an anti-cholesterol agent, 5) an insulin sensitizing agent, 6) a sweetening agent, 7) an aromatic agent, 8) an antioxidant agent, 9) an anti-inflammatory agent, 10) a dietary fiber, 11) an appetite control agent, and 12) a diuretic agent. Some embodiments may further provide a nutraceutical product that is effective in promoting weight loss. The nutraceutical product may include various combinations of active and inactive ingredients, wherein the final product promotes weight loss. In some instances, a nutraceutical beverage may be provided as a weight loss supplement that is ingested with a meal or between meals to suppress appetite and/or enhance metabolism. The various components of some embodiments of nutraceutical or weight loss beverage are discussed below.

### Weight Loss Beverage Component Overview

Stimulant Agent

Some weight loss beverages of the disclosed embodiments may include at least one stimulant agent. A stimulant agent is understood to include any naturally occurring or naturally derived component or composition that may stimulates the central nervous system, cardiovascular system and/or endrocrine system to increase the basal body metabolism. For example, in one embodiment a weight loss beverage may be provided which comprises naturally occurring caffeine.

Caffeine occurs naturally in a large number of plants. Caffeine may be added to the weight loss beverage in a purified or isolated form, or may be added as part of a natural product. For example, in some embodiments caffeine may be added to the weight loss beverage in the form of Jade Oolong tea. Oolong tea generally understood to include various types and/or mixtures of tea leafs which are grown and harvested in Taiwan, China, or other similar environments. The physical blends differ and therefore a naming convention has been established. Oolong tea types include Taiwanese Oolong tea, Min Lan Oolong tea, Dongding Oolong tea, Pouchong Oolong tea, Formosa Oolong, Chinese Oolong tea, Tie Guanyin Oolong tea, Alpine Oolong tea, Lishan Oolong tea, Dayulin Oolong tea, Ali Mountain Oolong tea, and Osmanthus Oolong tea.

In addition to comprising natural caffeine as a stimulant, Oolong tea includes additional benefits, such as fat burning properties, carbohydrate blocking properties, anti-cholesterol properties, and insulin sensitizing properties, each of which will be discussed below.

In some embodiments, a weight loss beverage may be provided comprising Oolong tea as a stimulant agent. In some formulations, a weight loss beverage may be provided comprising Oolong tea from approximately 0.01 grams per serving to approximately 3.0 grams per serving. In other formulations, a weight loss beverage may be provided comprising Oolong tea from approximately 0.05 grams per serving to approximately 2.0 grams per serving. Further, in some formulations a weight loss beverage may be provided comprising approximately 1.093 grams of Oolong tea per serving, wherein the Oolong tea comprises approximately 46.31% of the beverage serving, by weight.

In some embodiments, a weight loss beverage may be provided comprising a blend of Taiwanese Oolong tea and Min Lan Oolong tea. In some formulations, the weight loss beverage includes Taiwanese Oolong tea from approximately 0.5 grams per serving to approximately 1.5 grams per serving, and Min Lan Oolong tea from approximately 0.01 grams per serving to approximately 0.5 grams per serving. In other formulations, a weight loss beverage may be provided comprising Taiwanese Oolong tea from approximately 0.75 grams per serving to approximately 1.2 grams per serving, and Min lan Oolong tea from approximately 0.75 grams per serving to approximately 0.25 grams per serving. Further, in some formulations a weight loss beverage may be provided comprising approximately 0.983 grams of Taiwanese Oolong tea per serving and approximately 0.109 grams of Min Lan Oolong tea per serving, wherein the Taiwanese Oolong tea comprises approximately 41.68% of the beverage serving, by weight, and the Min Lan Oolong tea comprises approximately 4.63% of the beverage serving, by weight.

Fat Burning Benefits of Oolong Tea

As mentioned previously, one of the leading health benefits of Oolong tea may be its capacity to aid in weight loss by stimulating hormones that are responsible for the fat burning process. Hormones affect many different processes including metabolism, growth, development, mood and aging. “Fat burning” hormones are part of the endocrine system and may be stimulated by Oolong tea. Accordingly, some formulations of the disclosed embodiments comprise one or more types of Oolong tea for the purpose of promoting fat burning.

Carbohydrate Blocking Benefits of Oolong Tea

Further, Oolong tea has been shown to lower the body fat storage effects of carbohydrates. Specifically, Oolong tea has been shown to interfere with the body's ability to convert carbohydrate into glucose. By preventing the breakdown of carbohydrates into simple sugars, Oolong tea prevents the body from absorbing the carbohydrates. This results in weight loss due to reduced caloric intake. Accordingly, some formulations of the disclosed embodiments comprise one or more types of Oolong tea for the purpose of blocking carbohydrate metabolism.

Anti-Cholesterol Benefits of Oolong Tea

In some studies, Oolong tea has been shown to reduce high blood pressure and lower LDL cholesterol levels. Polyphenol antioxidants within the Oolong tea act to reduce inflammatory effects, such as coronary artery disease, and provide improved endothelial health via down-regulation of oxidative LDL cholesterol. Other long-term beneficial health effects resulting from consumption of foods rich in polyphenols (i.e. Oolong tea) may include anti-aging consequences, such as slowing the process of skin wrinkling. Accordingly, some formulations of the disclosed embodiments comprise one or more types of Oolong tea for the purpose of improving endothelial health.

Insulin Sensitizing Benefits of Oolong Tea

Further, some studies have shown that Oolong tea may be effective in lowering endogenous blood sugar levels. In particular, use of Oolong tea has demonstrated a reduction of serum glucose in a user by promoting increased sensitivity of the user to insulin. Excess blood sugar may be rapidly absorbed by the user's muscles thereby providing the user with increased energy and preventing conversion of the excess sugar into fat. Oolong tea may further provide or promote a reduction in triglycerides, LDL cholesterol, and total cholesterol levels in a user. Thus, some formulations of the disclosed embodiments comprise one or more types of Oolong tea for the purpose of enhancing insulin sensitivity in the user.

Some embodiments of the disclosed embodiments further include an insulin sensitizing agent comprising cinnamon bark. Cinnamon (Cinnamonum cassia) has been shown to reduce serum glucose, triglycerides, LDL cholesterol, and total cholesterol in people with type II diabetes. Thus, some formulations of the disclosed embodiments comprise a weight loss beverage containing cinnamon bark as an insulin sensitizing agent.

Additional spices including cloves, bay leaves, and turmeric further display insulin enhancing activity in vitro. Various botanical products have also been shown to improve glucose metabolism by hypoglycemic effect and also by improving lipid metabolism, antioxidant status, and blood capillary function. A number of medicinal/culinary herbs have also been reported to yield hypoglycemic effect in patients with diabetes. Examples of these include bitter melon, Gymnema, Korean ginseng, onions, garlic, flaxseed meal, and specific nutrients including α-lipoic acid, biotin, carnitine, vanadium, chromium, magnesium, zinc, and vitamins B₃, E, and K. Accordingly, various embodiments of the disclosed embodiments may include any component or combination of components which have been shown to act as an insulin sensitizing agent in accordance with the teachings of the disclosed embodiments.

Some embodiments may further include an insulin sensitizer comprising punicic acid. Some studies suggest that punicic acid provides an insulin sensitizing effect, wherein punicic acid acts as a ligand of PPARγ, which plays a crucial role in adipocyte differentiation. Accordingly, some weight loss compositions of the disclosed embodiments may utilize punicic acid as both an insulin sensitizing agent and a fat blocking agent.

Sweetening Agent

Some weight loss beverages of the disclosed embodiments comprise at least one sweetening agent. A sweetening agent may include any naturally occurring phytonutrient or phytochemical that may be capable of sweetening a weight loss beverage anticipated by the disclosed embodiments. In some embodiments, a sweetening agent comprises a low-carbohydrate, low-sugar additive.

For example, in some embodiments a sweetening agent comprises Stevia rebaudian, commonly known as sweetleaf, sugar leaf, or simply stevia (Rebaudioside A). As a sweetener and sugar substitute, stevia's taste has a slower onset and longer duration than that of sugar. Steviol glycoside extracts may be perceived as 300 times the sweetness of sugar. Additionally, stevia has negligible effect on blood glucose thereby making it a desirable sweetening agent for people on carbohydrate-controlled diets. In addition to being a sweetening agent, studies have found that stevioside and related compounds have anti-hyperglycemic, anti-hypertensive, anti-inflammatory, anti-tumor, anti-diarrheal, diuretic, and immunomodulatory actions. Thus, some formulations of the disclosed embodiments comprise a weight loss beverage containing stevia or a stevia derivative as a sweetening agent.

In some formulations, a weight loss beverage may be provided comprising stevia (Rebaudioside A) from approximately 0.0005 grams per serving to approximately 0.05 grams per serving. In other formulations, a weight loss beverage may be provided comprising stevia from approximately 0.001 grams per serving to approximately 0.01 grams per serving. Further, in some formulations a weight loss beverage may be provided comprising approximately 0.005 grams stevia per serving, wherein the stevia comprises approximately 0.21% of the beverage serving, by weight.

A weight loss beverage of the disclosed embodiments may further comprise inulin as a sweetening agent. Inulins are a group of naturally occurring polysaccharides produced by many types of plants. They belong to a class of dietary fibers known as fructans. Inulin may be used by some plants as a means of storing energy and is typically found in roots or rhizomes. Most plants that synthesize and store inulin do not store other forms of carbohydrates such as starch.

Inulin may be increasingly used in processed foods because it has unusually adaptable characteristics. Its flavor ranges from bland to subtly sweet (approximately 10% sweetness of sugar and/or sucrose). It can be used to replace sugar, fat and flour, and also provides fiber. This may be advantageous because inulin contains 25-35% of the food energy of carbohydrates (starch, sugar). In addition to being a versatile ingredient, inulin has many health benefits. For example, inulin increases calcium absorption and possible magnesium absorption, while promoting the growth of intestinal bacteria. In terms of nutrition, inulin may be considered a form of soluble fiber and may be sometimes categorized as a prebiotic. Due to the body's limited ability to process fructans, inulin has minimal increasing impact on blood glucose. Further, inulin is not insulemic and does not raise triglycerides, making it considered suitable for diabetics and potentially helpful in managing blood sugar-related illnesses. Thus, some formulations of the disclosed embodiments comprise a weight loss beverage containing inulin as a sweetening agent.

In some formulations, a weight loss beverage may be provided comprising inulin from approximately 0.05 grams per serving to approximately 1.5 gram per serving. In other formulations, a weight loss beverage may be provided comprising inulin from approximately 0.1 grams per serving to approximately 1.0 grams per serving. Further, in some formulations a weight loss beverage may be provided comprising approximately 0.645 grams of inulin per serving, wherein the inulin comprises approximately 27.33% of the beverage serving, by weight.

Aromatic Agent

Aromatherapy (i.e. a form of alternative medicine that uses essential oils and other aromatic compounds for the purpose of altering a person's mind, mood, cognitive function, and/or health) has been shown to affect brain chemistry related to appetite. The satiety response may be regulated in the ventromedial nucleus of the hypothalamus. This portion of the hypothalamus signals a feeling of fullness, or a sense of being satisfied with the amount of food eaten. Some studies have shown that scents or aromas play an important role in signaling to the hypothalamus that calories are being consumed by an individual. Thus, by stimulating the hypothalamus with aromas, the hypothalamus may be prompted to signal the feeling of fullness or satiety, thereby limiting overall caloric intake.

Aromatic agents of the disclosed embodiments may include any essential oil and/or fragrance that may be effective in stimulating the hypothalamus to signal a satiety response. Non-limiting examples of essential oils include allspice, bitter almond, anise, basil, caraway seed, cassia bark, ocotea leaf, ocotea quixos, celery leaves, celery seed, cilantro, cinnamon bark, cinnamon leaf, Clementine, clove bud, coriander seed, commint, cumin seed, black cumin, dill seed, dill weed, fennel, ginger, grapefruit, laurel leaf, lemon, lime, marjoram, mentha citrata, nutmeg, orange, parsley, black pepper, peppermint, rosemary, sage, sassafras, savory, spearmint, thyme, vanilla, and wintergreen. Non-limiting examples of fragrances include banana, chocolate, coffee, green apple, green tea, maple syrup, and peach. Accordingly, some formulations of the disclosed embodiments comprise a weight loss beverage containing one or more aromatic agents.

In some embodiments, a weight loss beverage may be provided comprising vanilla essential oil or vanilla extract (i.e., with a vanillin concentration over 200 ppm) as an aromatic agent. In some formulations, a weight loss beverage may be provided comprising vanilla essential oil from approximately 0.005 grams per serving to approximately 0.5 grams per serving. In other formulations, a weight loss beverage may be provided comprising vanilla essential oil from approximately 0.01 grams per serving to approximately 0.3 grams per serving. Further, in some formulations a weight loss beverage may be provided comprising approximately 0.15 grams of vanilla essential oil per serving, wherein the vanilla essential oil comprises approximately 6.36% of the beverage serving, by weight. In some embodiments, a weight loss beverage may further be provided comprising cinnamon bark as an aromatic agent.

Antioxidant Agent

Some weight loss beverages of the disclosed embodiments comprise at least one antioxidant agent. An antioxidant is a molecule that inhibits the oxidation of other molecules. Oxidation is a chemical reaction that transfers electrons or hydrogen from a substance to an oxidizing agent. Oxidation reactions can produce free radicals. In turn, these radicals can start chain reactions. When the chain reaction occurs in a cell, it can cause damage or death to the cell. Antioxidants terminate these chain reactions by removing free radical intermediates, and inhibit other oxidation reactions. They do this by being oxidized themselves into less reactive free radicals so antioxidants are often reducing agents such as thiols, ascorbic acid, polyphenols, or flavanoids.

Polyphenols are a broad class of naturally occurring antioxidants found in plants. Studies have found that polyphenols interfere with the function of human digestive enzymes. Specifically, polyphenols have been show to prevent the metabolism of dietary fats. As a result, polyphenols are effective in preventing the storage of unused, metabolized dietary fats as fatty tissue. Thus, the antioxidant properties of polyphenols provide a fat blocking function within the body.

Flavonoids are natural compounds found in many fruits, vegetables, and beverages like tea, coffee, beer, fruit juice, and red wine. Flavonoids are powerful antioxidants that can help fight infection, prevent inflammation, interfere with tumor growth, and effect metabolism of dietary fats. Non-limiting examples of flavonoids include quercetin, xanthohumol, isoxanthohumol, genistein, charlconaringenin, and naringenin.

Antioxidant agents in accordance with the disclosed embodiments include phytonutrients and/or phytochemicals high in polyphenols and/or flavonoids, including but not limited to cacao powder, blueberries, pomegranates, aronia berry, red grapes, cranberries, red or purple sweet potatoes, black rice, purple barley, black sorghum, hops, soy, citrus fruits, purple potatoes, apple cider, apple juice, coffee, tea, red wine, and yellow onions. Some antioxidant agents of the disclosed embodiments may further be included comprising high dietary fiber, magnesium, and vitamin C, which are effective for weight loss and overall health.

In some embodiments, a weight loss beverage may be provided comprising cacao powder (non-dutch, 10-12% fat) as an antioxidant agent. In some formulations, a weight loss beverage may be provided comprising cacao powder from approximately 0.01 grams per serving to approximately 1.0 grams per serving. In other formulations, a weight loss beverage may be provided comprising cacao powder from approximately 0.05 grams per serving to approximately 0.5 grams per serving. Further, in some formulations a weight loss beverage may be provided comprising approximately 0.175 grams of cacao powder per serving, wherein the cacao powder comprises approximately 7.41% of the beverage serving, by weight.

Anti-Inflammatory Agent

Some weight loss beverages of the disclosed embodiments comprise at least one anti-inflammatory agent. An anti-inflammatory agent refers to the properties of a substance that reduces inflammation. Some health professionals advocate the consumption of anti-inflammatory foods as a means of controlling inflammation and promoting health. Anti-inflammatory agents have been shown to improve overall health and improve digestion. Accordingly, some embodiments of the disclosed embodiments provide a weight loss beverage comprising one or more anti-inflammatory agents.

Anti-inflammatory agents in accordance with the disclosed embodiments may include phytonutrients and/or phytochemicals comprising various essential oils and plant-derived powders, including but not limited to thyme, fennel, cinnamon bark, bay leaf, clove, eucalyptus, bergamot, rose, peppermint, oregano, lemongrass, melaleuca ericifolia, nutmeg, dill, ledum, frankincense, basil, geranium, wintergreen, balsam fir, helichrysum, white fir, panaway, elemi, melaleuca alternifolia, lavender, myrrh, ravintsara, vanilla bean, vanilla, hyssop, copaiba, ocotea, palo santo, German chamomile, yarrow, blue tansy, BLM with MSM and glucosamine, and sulfurzyme with MSM, FOS and NingXia wolfberry. Anti-inflammatory agents of the disclosed embodiments may further comprise Ocotea leaf (Ocotea quixos) and its respective essential oil. Anti-inflammatory agents may further include synthetically derived anti-inflammatory compounds and/or agents as may be required to provide a desired benefit.

Some embodiments of the disclosed embodiments may include one or more anti-inflammatory agents comprising eugenol. Eugenol is a phenylpropene, an allyl chain-substituted guaiacol. Eugenol is a member of the phenylpropanoids class of chemical compounds. It is a clear to pale yellow oily liquid that may be extracted from certain essential oils, especially from clove oil, nutmeg, cinnamon, vanilla, basil, Japanese star anise, lemon balm, pimento racemosa, sweet basil, African basil, wormwood, cinnamomum tamala, allspice, carrot, laurel, majoram, pennyroyal, lavender, and bay leaf. Eugenol has been shown to have a calming effect on the brain and central nervous system, thereby suppressing the user's appetite and cravings for food. Eugenol has also been shown effective in relieving anxiety, anger and restlessness, thus further diverting conditions that may drive the user to consume food. Eugenol operates by inhibiting nerve transmission between cells and the synthesis of prostaglandins. Thus, some formulations of the disclosed embodiments may incorporate one or more anti-inflammatory agents comprising eugenol for the purpose of reducing inflammation, anxiety and other related indications.

In some embodiments, a weight loss beverage may be provided comprising frankincense powder as an anti-inflammatory agent. Frankincense, also called olibanum, is an aromatic resin obtained from tress of the genus Boswellia, particularly Boswellia sacra, Boswellia carterii, Boswellia thurifera, Boswellia frereana, Boswellia serrata, Boswellia papyrifera, and Boswellia bhaw-dajiana (Burseraceae). These Boswellia species have been shown to include natural anti-inflammatory components with boswellic acids as an example. There are four main species of Boswellia which produce true frankincense, and each type of resin is available in various grades, including, but not limited to Silver, Hojari, and Maydi.

In some formulations, a weight loss beverage may be provided comprising frankincense powder from approximately 0.005 grams per serving to approximately 0.2 grams per serving. In other formulations, a weight loss beverage may be provided comprising frankincense powder from approximately 0.01 grams per serving to approximately 0.075 grams per serving. Further, in some formulations a weight loss beverage may be provided comprising approximately 0.025 grams of frankincense powder per serving, wherein the frankincense powder comprises approximately 1.06% of the beverage serving, by weight.

In some embodiments, a weight loss beverage may be provided comprising vanilla essential oil as an anti-inflammatory agent. In some formulations, a weight loss beverage may be provided comprising vanilla essential oil from approximately 0.005 grams per serving to approximately 0.5 grams per serving. In other formulations, a weight loss beverage may be provided comprising vanilla essential oil from approximately 0.01 grams per serving to approximately 0.3 grams per serving. Further, in some formulations a weight loss beverage may be provided comprising approximately 0.15 grams of vanilla essential oil per serving, wherein the vanilla essential oil comprises approximately 6.36% of the beverage serving, by weight. In some embodiments, a weight loss beverage may further be provided comprising cinnamon bark and/or ground nutmeg as an anti-inflammatory agent.

In some embodiments, a weight loss beverage may be provided comprising Ocotea Leaf (Ocotea quixos) as an anti-inflammatory agent. Ocotea quixos is a species of evergreen tree in the Ocotea genus of plants in the Lauraceae family. It is commonly found in Ecuador and Colombia.

Ocotea quixos is also known as “Ecuadorian cinnamon” or “False cinnamon.” The cinnamon-like scent and flavor is attributed to the presence of methyl cinnamate (15-35%%) and trans-cinnamaldehyde (10-30%) which are also found in the essential oils which are derived from the flower calices of the Ocotea quixos plant. Studies of trans-cinnamaldehyde have shown significant reduction in inflammation or the inflammatory response. In particular, studies have linked trans-cinnamaldehyde to the reduced production of nitrous oxide by macrophages, an action typically associated with the inflammatory response. Accordingly, in some embodiments a weight loss beverage may be provided comprising Ocotea quixos as an anti-inflammatory agent. Further, in some embodiments a weight loss beverage may be provided comprising Ocotea quixos as an aromatic agent.

For example, in some formulations a weight loss beverage may be provided comprising ocotea leaf (Ocotea quixos) from approximately 0.05 grams per serving to approximately 0.8 grams per serving, assuming a 1.9% yield of essential oil from the dried ocotea leaf. In other formulations, a weight loss beverage may be provided comprising ocotea leaf from approximately 0.128 grams per serving to approximately 0.38 grams per serving. Further, in some formulations a weight loss beverage may be provided comprising approximately 0.267 grams of ocotea leaf per serving, wherein the ocotea leaf comprises approximately 11.31% of the beverage serving, by weight.

Dietary Fiber

Some weight loss beverages of the disclosed embodiments may comprise one or more dietary fibers. Dietary fiber, also known as roughage or bulk, includes all parts of plant food that the body may be incapable of digesting. Unlike other food components such as fats, protein and/or carbohydrates, fiber is not readily digested by the body. Therefore, fiber passes through the digestive system relatively intact. Fiber is commonly classified into two categories, namely insoluble fiber and soluble fiber. Inulin may also be used as a dietary fiber.

A high fiber diet has been shown to slow the absorption of sugar by the body. Thus, fiber functions to help control blood sugar levels and reduce the amount of sugar metabolized by the body. Dietary fibers in accordance with the disclosed embodiments may comprise phytonutrients and phytochemicals including, but not limited to cacao powder, ground nutmeg, cinnamon bark, and inulin. Weight loss beverages in accordance with the disclosed embodiments may generally include one or more dietary fibers as described herein. Amounts and ranges of dietary fibers for weight loss beverages of the disclosed embodiments may have been previously discussed. Some weight loss beverages may further include any known dietary fiber that may be affective in slowing the absorption of sugar by the body.

Diuretic Agent

Some weight loss beverages may comprise at least one diuretic agent. A diuretic agent may be understood to include any phytonutrient and/or phytochemical which provides a means of forced diuresis which elevates the rate of urination. Diuretic agents in accordance with the disclosed embodiments may include frankincense powder, as discussed herein. Accordingly, some formulations of the disclosed embodiments may provide a weight loss beverage which includes frankincense powder as a diuretic. Amounts and ranges of diuretic agents for weight loss beverages of the disclosed embodiments may have been discussed previously. Weight loss beverages in accordance with the disclosed embodiments may further include any known diuretic agent that may be affective in providing forced diuresis.

Appetite Control Agent

Some weight loss beverages may comprise at least one appetite control agent. An appetite control agent may be understood to be any compound that decreases appetite. For example, natural compounds in cinnamon and Oolong components may each have the effect of decreasing ghrelin, a hunger hormone, and increasing leptin and adiponectin, which may aid in controlling hunger pangs and impulses. Other agents may be selected which provide an appetite control effect.

Other Natural Active Ingredients

Any suitable essential oil can be employed in a weight loss beverage provided (1) the essential oil has therapeutic properties (e.g., the essential oil may be effective in promoting weight loss), (2) the essential oil remains thermally stable in the composition, and (3) the essential oil is non-toxic to mammals (e.g., humans) and will be suitable for oral administration. Preferably, the thermostability and photostability of the essential oil is over a prolonged period of time, e.g., up to about 3 years, up to about 1 year, or up to about 6 months, typically experienced in the manufacturing, packaging, shipping, and/or storage of the composition. Included essential oil may also preferably comply with any controlling or governing body of law.

Suitable specific essential oils may be derived from and include one or more of the following: ajowan, sweet almond, allspice, aloe vera, ammi visnaga (khella), amyris, angelica root, angelica seed, anise, anise seed, star anise, apricot kernel, absolute arnica, avocado, unrefined avocado, Copaiba balsam, balsam Peru genuine, balsam Peru, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel, clove, clove bud, clove leaf, cocoa, cocoa butter, unrefined cocoa butter, coconut, refined coconut, cognac, combava petitgrain, coriander, green coriander, cornmint, costus, cumin, cypress, davana, dill, dill weed, elemi, erigeron (fleabane), eucalyptus citriodora, eucalyptus globulus, lemon eucalyptus, fennel, sweet fennel, fenugreek, fir (i.e. abies ssp.), fir needles (i.e. Canada fir needle, Siberia fir needle, white fir needle, etc.), frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed, hazelnut, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper (i.e. juniperus ssp), juniper berry (i.e, Siberia juniper berry, Croatia juniper berry, etc.), lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavender, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, ocotea, olibanum, absolute opopanax, bitter orange, blood orange, sweet orange, wild West Indian orange, oregano, orris root, concrete orris, osmanthus, palm, refined palm, palmarosa, paprika, parsley seed, patchouli, Indian patchouli oil, Indonesian patchouli, peanut, pecan, pennyroyal, pepper, black pepper, super black pepper, peppermint, India peppermint, USA baby mint peppermint, petitgrain (orange leaves), alcoquiana, pine (i.e. pinus ssp), pine needle (i.e., white pine needle, etc.), evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce (i.e., picea ssp.), St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra. Specifically, suitable exemplary essential oils may include citral, cinnamon cassia, ocotea, pomegranate seed oil, or a combination thereof.

Other essential oils and additives may include, laurus nobilis, melaleuca alternifolia, Melaleuca quienquenervia, Melaleuca ericifolia, hibiscus abelmoschus, beeswax absolute (apis mellifera), bergamot mint (mentha citrate), bougoin de cassis (ribes nigrum), broom absolute (spartium junceum), cajuput (melaleuca leucadendron), camphor (Cinnamomum camphora), cardamon seed (Ellettaria cardamomum), cassie absolute (Acacia farnesiana), everlasting absolute (Helichrysum stoechas), hay absolute, ho wood (Cinnamomum camphora), labdanum absolute (Cistus ladaniferus), lentisque absolute (Pistacia lentiscus), orange flower absolute (Citrus aurantium), origanum, savory (Satureja montana), spearmint (Mentha spicata), tobacco absolute (spp. nicotiana), tonka absolute (Dipteryx odorata), treemoss absolute (Evernia prunastri), vetyvert (Vetivera zizanoides), d-limonene, 1-Limonene, myrcene, cineole, menthol, menthone, menthyl acetate, alpha-humulene, gamma-humulene, caryophyllene, borneol, linalool, linalyl acetate, methyl salicylate, bornyl acetate, para-cymene, eugenal, geraniol, nerol, citronellol, sabinene, alpha-pinene, beta-pinene, eudesmol, aromadendrebe, globuluol, terpinen-4-ol, terpinolene, cuminal aldehyde, alpha-terpineol, bisaboilol, bisabolol oxides, ocimene, myrcene, fenchol, germacrene D, C, B, a zingiberene, gamma-cadinene, beta-selinene, farnesol, delta-cadinene, alpha-selinene, beta-selinene, gamma-selinene, delta-elemene, alpha-elemene, piperine, carvone, benzaldehyde, anisyl acetate, anisyl alchohol, camphene, geranyl acetate, isomenthol, isomenthone, vanillin, terpineol, valencene, sinensal, nootketone, gamma-terpinene, thymol, carvacrol, alpha-bergamotene, cubebol, merolidol, neryl acetate, methyl eugenol, longifolene, anethole, anisyl acetate, benzyl benzoate, benzyl cinnamate, 5-epiprezizane, khusimene, alpha-muurolene, khusimone, calacorene, beta-humulene, alpha-longipinene, delta-cadinene, valencene, calarene-gurjunene, alpha-amorphene, epizizanal, 3-epizizanol, khusimol, isoeugenol, alpha-curcumene, beta-curcumene, gamma-curcumene, perrilyl alcohol, iso-khusimol, valerenol, beta-vetivone, alpha-vetivone, Ethyl-2-Methylbutyrate, Isoamyl Isovalerate, Isopropyl-2-Methylbutyrate, 2-Methylbutyric Acid, Methyl-2-Methylbutyrate, Methyl Isovalerate, Phenylethyl Isovalerate, Benzyl Butyrate, Benzyl Isobutyrate, Benzyl Isovalerate, Benzyl Propionate, Butyl Butyrate, Ethyl Isovalerate, Ethyl Valerate, n-Hexyl Isobutyrate, n-Hexyl-2-Methylbutyrate, Isoamyl-2-Methylbutyrate, Isobutyl-2-Methylbutyrate, Phenethyl Butyrate, Phenethyl Isobutyrate, Phenethyl-2-Methylbutyrate, or any combination thereof.

The amount of essential oils in the weight loss beverage may be within the effective ranges of the individual oils. For example, in one embodiment a single essential oil may be added to the beverage within an effective range of 500 ppm to 3500 ppm. In another embodiment, a first essential oil may be added to the beverage within an effective range of 500 ppm to 3500 ppm, and a second essential oil may be added to the beverage within an effective range of 100 ppm to 1500 ppm. In another embodiment, a first essential oils may be added to the beverage within an effective range of 500 ppm to 3500 ppm, a second essential oil may be added to the beverage within an effective range of 50 ppm to 1500 ppm, and a third essential oil may be added to the beverage within an effective range of 50 ppm to 1500 ppm. In another embodiment, a first and second essential oil may be added to the beverage within an effective range of 500 ppm to 3500 ppm, and a third essential oil may be added to the beverage within an effective range of 50 ppm to 1500 ppm. In yet another embodiment, a first, a second, and a third essential oil may be added to the beverage within an effective range of 500 ppm to 3500 ppm, and a fourth essential oil may be added to the beverage within an effective range of 50 ppm to 1500 ppm. Finally, in yet another embodiment, one or more essential oils are added to the beverage within an effective range of 500 ppm to 3500 ppm, and one or more additional essential oils are added to the beverage within an effective range of 50 ppm to 1500 ppm.

Weight loss beverages can be produced by a variety of methods. In some embodiments, an effective amount of each of the components described above are preformulated as separate ingredients. Thereafter, the separate ingredients may be mixed in effective ratios, as discussed above. Any optionally added ingredients, such as flavorings, dietary supplements, rheology modifiers, emulsifiers, solvents, colorants, and the like may be preferably added according to the ratios discussed above.

### Weight Loss Beverage

Some embodiments of a weight loss beverage may include a tea mixture which includes various selections of the ingredients discussed above. In some embodiment, the weight loss beverage may include Slique Tea (mfg. by Young Living Essential Oils, LC. in Lehi, Utah). An additional representative weight loss beverage is provided in Example 1, below.

### EXAMPLES

### Example 1

### Weight Loss Beverage

Some embodiments may include a weight loss beverage comprising a serving size of approximately 2.36 grams, wherein each serving includes approximately 1.093 grams of Oolong tea (the Oolong tea comprising approximately 0.983 grams of Taiwanese Tea (#67/equivalent (ABC)), and approximately 0.109 grams of Min Lan tea (ABC)), approximately 0.645 grams of Inulin, approximately 0.175 grams of Cacao powder (Guittard, Non-Dutch, 10-12% fat), approximately 0.15 grams of Vanilla essential oil (16× concentration, Brazil), approximately 0.025 grams of Frankincense powder , approximately 0.267 grams of ocotea leaf (Ocotea quixos), and approximately 0.005 grams of Stevia (Rebaudioside A).

### Example 2

### Method of Use

A method for administering a weight loss beverage may be as follows. The weight loss beverage may be added to eight ounces of boiling water and allowed to steep for approximately three minutes. The user may then add one or more essential oils as a flavorant. The weight loss beverage may be taken by the user daily before and after workouts, with meals, and as needed to provide the user with additional energy and at times to reduce the user's appetite.

In addition to the disclosed embodiments, other embodiments may be employed in other specific forms without departing from its structures, methods, or other essential characteristics as broadly described and claimed. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed is:

1. A beverage composition for promoting weight loss, comprising:
a stimulant agent;
a fat burning agent;
a carbohydrate blocking agent;
an anti-cholesterol agent;
an insulin sensitizing agent;
a sweetening agent;
an aromatic agent;
an antioxidant agent;
an anti-inflammatory agent;
a dietary fiber; and
a diuretic agent.

2. The beverage composition of claim 1, wherein the stimulant agent is Oolong tea.

3. The beverage composition of claim 2, wherein Oolong tea comprises a mixture of Taiwanese tea and Min Lan tea.

4. The beverage composition of claim 1, wherein the fat burning agent, the carbohydrate blocking agent, the anti-cholesterol agent, and the insulin sensitizing agent is Oolong tea.

5. The beverage composition of claim 1, wherein the sweetening agent is selected from the group consisting of stevia and inulin.

6. The beverage composition of claim 1, wherein the aromatic agent is selected from the group consisting of vanilla essential oil, and ocotea leaf.

7. The beverage composition of claim 1, wherein the antioxidant agent is cacao powder.

8. The beverage composition of claim 1, wherein the anti-inflammatory agent is selected from the group consisting of frankincense powder, vanilla essential oil, and ocotea leaf.

9. The beverage composition of claim 8, wherein the frankincense powder is derived from at least one of Boswellia sacra, Boswellia carterii, Boswellia thurifera, Boswellia frereana, and Boswelli bhaw-dajiana.

10. The beverage composition of claim 1, wherein the dietary fiber is selected from the group consisting of cacao powder and inulin.

11. The beverage composition of claim 1, wherein the diuretic agent is frankincense powder.

12. A method for promoting weight loss, the method comprising:
providing a beverage comprising:
a stimulant agent;
a fat burning agent;
a carbohydrate blocking agent;
an anti-cholesterol agent;
an insulin sensitizing agent;
a sweetening agent;
an aromatic agent;
an antioxidant agent;
an anti-inflammatory agent;
a dietary fiber; and
a diuretic agent; and
administering the beverage to a user before meals, with meals, before workouts, after workouts, or any combination thereof.

13. The method of claim 12, wherein the stimulant agent, the fat burning agent, the carbohydrate blocking agent, the anti-cholesterol agent, and the insulin sensitizing agent is Oolong tea.

14. The method of claim 13, wherein Oolong tea comprises a mixture of Taiwanese tea and Min Lan tea.

15. The method of claim 14, wherein the Oolong tea comprises approximately 46.31% of a serving of the beverage, by weight.

16. The method of claim 12, wherein the sweetening agent is inulin, and wherein inulin comprises approximately 27.33% of a serving of the beverage, by weight.

17. The method of claim 12, wherein the sweetening agent is stevia, and wherein stevia comprises approximately 0.21% of a serving of the beverage, by weight.

18. The method of claim 12, wherein the antioxidant agent is cacao powder, and wherein cacao powder comprises approximately 7.41% of a serving of the beverage, by weight.

19. The method of claim 12, wherein the anti-inflammatory agent is selected from the group consisting of frankincense powder, vanilla essential oil, and ocotea leaf.

20. A beverage composition for promoting weight loss, comprising:
Oolong tea from approximately 0.01 grams per serving to approximately 3.0 grams per serving;
stevia from approximately 0.0005 grams per serving to approximately 0.05 grams per serving;
inulin from approximately 0.05 grams per serving to approximately 1.5 grams per serving;
vanilla essential oil from approximately 0.005 grams per serving to approximately 0.5 grams per serving;
cacao powder from approximately 0.01 grams per serving to approximately 1.0 grams per serving;
frankincense powder from approximately 0.005 grams per serving to approximately 0.2 grams per serving; and
ocotea leaf from approximately 0.05 grams per serving to approximately 0.8 grams per serving.
